(19) United States
(12) Patent Application Publication
(10) Pub. No.: US 2024/0268964 A1
(43) Pub. Date: Aug. 15, 2024

(54) TIBIA COMPONENT AND KNEE-JOINT ENDOPROSTHESIS SYSTEM

(71) Applicant: Aesculap AG, Tuttlingen (DE)

(72) Inventor: Thorsten Ketterer, Konstanz (DE)

(21) Appl. No.: 18/643,082

(22) Filed: Apr. 23, 2024

Related U.S. Application Data
(63) Continuation of application No. PCT/EP2022/079885, filed on Oct. 26, 2022.

(30) Foreign Application Priority Data
Oct. 26, 2021 (DE) 10 2021 127 834.1

(51) Int. Cl. A61F 2/38; A61B 34/20

(52) U.S. Cl. CPC A61F 2/389; A61B 34/20; A61F 2/3859; A61F 2002/3881

(57) ABSTRACT

A tibial component includes a tibial plate having a top side and a bottom side. A shaft projects from the bottom side for insertion into the medullary space of a tibia for anchoring the tibial component to the tibia. The tibial component includes a positioning device for positioning, in particular steplessly positioning, the shaft on the tibial plate in a plurality of different positions. The positioning device is configured in such a way that the shaft is transferable from one of the different positions into another one of the different positions by a movement in parallel to a positioning plane, which extends in parallel or substantially in parallel to the top side. The tibial component can be part of a knee joint endoprosthesis system.

### CROSS-REFERENCE TO RELATED APPLICATION(S)

This application is a continuation of International Application No. PCT/EP2022/079885, filed on Oct. 26, 2022, which claims priority to German Application No. 10 2021 127 834.1, filed on Oct. 26, 2021. The contents of International Application No. PCT/EP2022/079885 and German Application No. 10 2021 127 834.1 are incorporated by reference herein in their entireties.

### FIELD

The present disclosure relates to a tibial component for a knee joint endoprosthesis, wherein the tibial component comprises a tibial plate with a top side and a bottom side and comprises a shaft projecting from the bottom side for insertion into the medullary space of a tibia for anchoring the tibial component to the tibia, wherein the tibial component comprises a positioning device for positioning, in particular steplessly positioning, the shaft on the tibial plate in a plurality of different positions.

The present disclosure further relates to a knee joint endoprosthesis system with at least one femoral component for anchoring to a distal end of a femur and at least one tibial component for anchoring to a proximal end of a tibia, wherein the at least one femoral component and the at least one tibial component are configured corresponding to one another to form a knee joint endoprosthesis.

### BACKGROUND

Knee joint endoprosthesis systems of the kind described at the outset are used to replace damaged knee joints of patients by implanting a knee joint endoprosthesis suitable for the patient. Despite the wide variety of prosthesis types, there are still a not negligible number of patients in whom the tibial plate does not sit optimally on the tibia after implantation. In particular, there may be a protrusion of the tibial plate on one side on the tibia. As a result, patients are dissatisfied with the outcome of the implantation, as the tibial plate is immediately and unpleasantly noticeable to them as an edge.

### SUMMARY

It is therefore an object of the present disclosure to improve a tibial component and a knee joint endoprosthesis system of the kind described at the outset in such a way that, in particular, better surgical outcomes are made possible.

This object is achieved, in accordance with the present disclosure, in a tibial component of the kind described at the outset in that the positioning device is configured in such a way that the shaft is transferable from one of the different positions into another one of the different positions by a movement in parallel to a positioning plane, which extends in parallel or substantially in parallel to the top side.

The proposed further development of a known tibial component makes it possible, in particular, to move the shaft practically in parallel to itself relative to the tibial plate, i.e. by a movement in parallel to the positioning plane. This allows the shaft to be optimally arranged or positioned on the tibial plate for the patient's respective physiological situation. This is particularly advantageous when a medullary canal of the tibia into which the shaft must be inserted is not aligned completely symmetrically with respect to the prepared tibial face against which the tibial plate is brought into abutment. Thus, with the improved positioning device, it is possible, in particular, to optimally cover the tibial top side prepared by partial resection with the tibial plate, even if the medullary canal is not formed centrally relative to this face. A surgeon can then first, for example, position the shaft in the medullary canal and align the tibial plate relative to the shaft, so that an optimal adaptation of the tibial component to the tibia is possible.

It is favorable if the tibial component comprises a fixing device for fixing the shaft in an implantation position and if the fixing device is transferable from an alignment position, in which the shaft is movable, in particular displaceable, relative to the tibial plate in parallel to the positioning plane, and, in particular, alignable relative to the tibial plate, into an implantation position, in which the shaft is immovably held on the tibial plate. The fixing device thus makes it possible, in particular, to adjust the tibial component in the alignment position in the desired manner, i.e. to specify a position of the shaft relative to the tibial plate in the desired manner, and to then to fix this position temporarily or permanently by transferring the fixing device from the alignment position into the implantation position. The shaft is then immovably held on the tibial plate. In particular, a stable connection between the tibial component and the tibia can thus be established.

It is advantageous if the positioning device comprises a positioning element receptacle formed on the tibial plate and a positioning element arranged in the positioning element receptacle, if the positioning element and the shaft are configured to be coupleable to one another, and if the positioning element receptacle defines a longitudinal axis running perpendicularly to the positioning plane and is configured and dimensioned in such a way that the positioning element in the alignment position is displaceable relative to the positioning element receptacle in parallel to the positioning plane. The proposed further development makes it possible, in particular, to displace the shaft in the alignment position in parallel to the positioning plane relative to the tibial plate. In particular, a modular configuration of the tibial component is also possible in this way, because the positioning element and the shaft are configured to be coupleable to one another. For example, shafts of different lengths can be coupled to the positioning element, depending on which shaft length is optimal for a patient. In this case, the shaft does not need to be configured in a particular way for the desired positioning. The positioning of the shaft is determined by a relative position of the positioning element and positioning element receptacle relative to one another.

It is favorable if the positioning element receptacle is configured and dimensioned in such a way that the positioning element is secured in each of the different positions against a movement away from the tibial plate in a direction pointing away from the bottom side. This is advantageous, in particular, if the positioning element receptacle comprises a perforation. It can thus be prevented, for example, that the positioning element is able to pass through the perforation, and thus leave the positioning element receptacle in a direction away from the bottom side. The proposed configuration thus makes it possible to secure the positioning element in the positioning element receptacle in a defined manner.

It is advantageous if the positioning device comprises a movement delimiting device for delimiting a movement of the positioning element relative to the positioning element receptacle in a direction transverse, in particular perpendicular, to the top side away from the bottom side. The movement delimiting device thus prevents, in particular, that the positioning element is able to move in a direction away from the bottom side.

The position device can be configured in a simple manner if a perforation is formed on the tibial plate, if the perforation comprises the positioning element receptacle, and if the perforation comprises a stop that cooperates with the positioning element and acts in the direction toward the top side. With the perforation, it is possible, in particular, to couple the positioning element to a shaft. In particular, the stop can prevent that the positioning element is able to pass through the perforation. The stop retains the positioning element in the positioning element receptacle, namely in each of the different positions that the positioning element may adopt relative to the positioning element receptacle or in said positioning element receptacle.

It is advantageous if the positioning element receptacle is delimited at the bottom side by a wall and if the perforation comprises a receiving portion defining the positioning element receptacle and a perforation portion passing through the wall. This configuration makes it possible, in particular, to accommodate the positioning element in the positioning element receptacle in the region of the receiving portion. Furthermore, the positioning element passes through or engages at least partially into the perforation portion, for example with a projection. A delimitation of a movement of the positioning element in the positioning element receptacle in parallel to the positioning plane can then be predetermined selectively by the cooperation of the positioning element and the receiving portion or the positioning element and the perforation portion.

It is favorable if a cross sectional area of the receiving portion relative to the longitudinal axis is greater than a cross sectional area of the perforation portion. This makes it possible, in particular, to displace the positioning element in the positioning element receptacle in parallel to the positioning plane without it being able to pass through the perforation, in particular through the perforation portion.

The positioning device can be configured in a simple manner if the wall forms the stop. In particular, the wall can thus form a retaining flange for the positioning element, said retaining flange defining a planar stop face.

The wall favorably defines an annular stop face pointing in the direction toward the top side. Annular does not necessarily mean circular here. Annular means, in particular, an annular, self-contained face. In particular, the perforation portion may define a circular, oval, or polygonal, in particular quadrilateral, cross sectional area. The annular stop face, which, in particular, may be of planar configuration, cooperates with a side face of the positioning element pointing away from the bottom side in order to secure said positioning element against a movement through the perforation.

It is advantageous if the positioning element comprises a positioning element projection, which engages into or passes through the perforation portion. The positioning element projection can serve, in particular, to delimit a movement of the positioning element relative to the positioning element receptacle in parallel to the positioning plane. In particular, the positioning element projection can thus cooperate with the perforation portion, which mutually define stops for delimiting a relative movement in parallel to the positioning plane.

It is favorable if a cross sectional area of the positioning element projection relative to the longitudinal axis is smaller than a cross sectional area of the perforation portion. This allows the positioning element to move in the positioning element receptacle in parallel to the positioning plane, namely within the cross sectional area defined by the perforation portion. The movement is possible in all directions in the positioning plane, but only until the positioning element projection strikes against the perforation portion.

A first cross sectional shape of the receiving portion and a second cross sectional shape of the perforation portion are advantageously geometrically similar or differ from one another. If the receiving portion and the perforation portion are geometrically similar and, for example, of circular configuration, the positioning element can, in particular, also be rotated in the positioning element receptacle. If the receiving portion and the perforation portion differ in their cross sectional shape, it is possible, in particular, to restrict a rotation of the positioning element in the positioning element receptacle in a defined manner. For example, a positioning element projection, which is polygonal, can cooperate with a corresponding perforation portion, the second cross sectional shape of which differs from the cross sectional shape of the positioning element projection.

The first and/or the second cross sectional shape is/are preferably oval, in particular circular, or polygonal, in particular rectangular or square. By selecting the appropriate cross sectional shapes, movements of the positioning element and the positioning element receptacle can be restricted in a desired manner, as already described above.

In accordance with a further preferred embodiment, provision may be made that the fixing device is configured to immovably fix the positioning element in the positioning element receptacle in the implantation position. With such a fixing device, it is possible, in particular, to position the positioning element immovably on the tibial plate and thus a shaft coupled to the positioning element.

It is advantageous if the fixing device comprises a fixing element and if the fixing element abuts directly or indirectly against the positioning element and in the implantation position holds the positioning element in the positioning element receptacle in a clamping manner. Such a configuration can be achieved in a simple manner, for example by a fixing element in the form of a fixing screw or a fixing nut.

Preferably, the positioning element in the implantation position is held between the fixing element and the stop in a clamping manner. With such a configuration, in particular, a number of parts required for the formation of the tibial component can be minimized.

It is favorable if the fixing element comprises a screw element with an external thread and if the perforation comprises an internal thread corresponding to the external thread commencing from the top side of the tibial plate. Such a configuration makes it possible, in particular, in a simple manner to transfer the positioning element from the alignment position into the implantation position, namely by the fixing element being screwed into the perforation until, in particular, the positioning element is held between the fixing element and the stop in a clamping manner. In addition, a surgeon can thus fix the position of the positioning element in a simple manner when the shaft is already inserted into the medullary space and the tibial plate is placed against the prepared tibial face. Only when the shaft and tibial plate are aligned relative to one another in the desired manner can a surgeon quickly and securely transfer the tibial component from the alignment position into the implantation position.

In the implantation position, the fixing element preferably closes the perforation. In particular, a top side of the tibial plate that is closed throughout can thus be achieved.

It is favorable if the fixing element has a fixing element clamping face, which in the implantation position is held against the positioning element in a clamping manner. In particular, the fixing element clamping face may be rough or have a surface structure in order in order to prevent a relative movement of the positioning element relative to the fixing element in a direction parallel to the positioning plane.

It is advantageous if the positioning element has an, in particular planar, fixing element abutment face, which in the implantation position abuts against the fixing element. In particular, an optimal clamping between the fixing element and the positioning element in the implantation position can thus be achieved.

On the tibial plate, the shaft can project from the tibial plate, in particular, perpendicularly or at a defined, unchangeable angle. In particular, it can project from the positioning element in this manner. In order to enable an optimal alignment of the shaft relative to the tibial plate, it is favorable if the shaft is mounted on the tibial plate so as to be pivotable and/or so as to be rotatable about a longitudinal axis of the shaft. In particular, this configuration enables a desired alignment of the shaft relative to the tibial plate. For example, the shaft may be mounted on the tibial plate so as to be steplessly pivotable and/or rotatable. Alternatively, it is also possible, in particular, to predetermine a multitude of defined pivot and/or rotation positions of the shaft relative to the tibial plate, i.e. a detented pivoting or detented rotation. This enables a multitude of different orientations of the shaft relative to the tibial plate, but not in a stepless manner. As a result of the proposed special mounting of the shaft on the tibial plate, it is possible, in particular, to always optimally align the shaft so that it can be inserted anatomically correctly into the medullary space of the tibia of the patient. In particular, a conflict between the shaft and a bone face of the tibia delimiting the medullary space can thus be prevented. In particular, a shaft of the tibial component can thus be oriented in any manner both in the anterior-posterior direction and in the medial-lateral direction. Thus, in particular, stress peaks, which are caused by a pressure or an edge load of the shaft in the medullary space on the inner cortical of the tibia, can be minimized. Due to the described ability to optimally align the shaft of the tibial component, in particular a so-called kinematic alignment of the knee joint endoprosthesis is able to be achieved at all, namely in each patient. In particular, this makes it possible to maintain a natural stable capsule-ligament tension without the need to reduce ligament tension by partially severing ligaments on the patient's knee. This so-called “release” is therefore not necessary.

The shaft is favorably arranged or formed in the region of a symmetry plane of the tibial plate. Due to this configuration of the positioning device, the shaft can, in particular, be moved in the symmetry plane or be moved out of it, depending on what is required for the patient.

It is advantageous if the shaft is arranged or formed in a middle or central region of the bottom side of the tibial plate. A basic position of the shaft relative to the tibial plate can thus be predetermined. Starting from this basic position, an adjustment of the shaft or an alignment thereof relative to the tibial plate can then be performed depending on the physiology of the patient.

The tibial component can be formed in a simple manner if the shaft is of rotationally symmetrical or substantially rotationally symmetrical configuration relative to is longitudinal axis.

The shaft can be pivoted relative to the tibial plate or be rotated about its longitudinal axis in a simple manner if it is mounted on the tibial plate in an articulated manner. In particular, it may be mounted in a hinge-jointed or ball-jointed manner. In addition to the displaceability of the shaft relative to the tibial plate, such a bearing makes it possible, in particular, to pivot said shaft relative to the tibial plate.

In accordance with a further preferred embodiment, provision may be made that the tibial component comprises a joint device with a first joint element and a second joint element, if the first joint element is arranged or formed on the tibial plate, and if the second joint element is arranged or formed on the shaft, and if the first joint element and the second joint element are in engagement with one another in an articulated manner. Such a joint device enables, in particular, a defined pivoting and/or rotation of the joint elements of the joint device that are in engagement with and cooperate with one another. In particular, the joint device may be configured to establish a hinge-jointed or a ball-jointed connection between the joint elements.

Preferably, the first joint element is configured in the form of a joint receptacle and the second element in the form of a joint projection engaging into the joint receptacle. A joint device configured in that way can be produced in a simple manner and enables, in particular, a flexible alignment of the shaft relative to the tibial plate.

It is favorable if the joint projection is of spherical configuration and if the joint receptacle has a hollow-spherical abutment face for the joint projection. This configuration makes it possible, in particular, in a simple manner to produce a ball-jointed connection with the cooperating joint elements. A shaft with a joint ball can thus be rotated in a simple manner by 360° relative to its longitudinal axis in the joint receptacle and also be pivoted relative to a surface normal of the bottom side of the tibial plate, namely preferably within a predetermined angular range. This angular range may be delimited, in particular, by suitable stops on the shaft and/or on the tibial plate.

The positioning device can be configured in a particularly compact manner if the positioning element comprises the joint receptacle.

It is advantageous if the positioning element is of two-part configuration and comprises a first positioning element part and a second positioning element part, if the first positioning element part has a joint projection seat for the joint projection, and if the second positioning element part is arranged between the first positioning element part and the fixing element and abuts against the joint projection. Such a configuration makes it possible, in particular, in a simple manner to fix a shaft articulatedly mounted on the positioning element in the implantation position, in particular by clamping the joint projection between the two positioning element parts.

Preferably, the second positioning element part comprises the fixing element abutment face. In particular, said fixing element abutment face may be adapted to a contour of the joint projection, so that it always abuts against the joint projection in a defined manner.

The tibial component can be formed in a simple manner if the joint receptacle is of rotationally symmetrical configuration. In particular, the joint receptacle may be of rotationally symmetrical configuration relative to a surface normal of the bottom side of the tibial plate.

In accordance with a further preferred embodiment, provision may be made that the shaft is of multi-part configuration and comprises a first shaft component and a second shaft component, that the first shaft component in the implantation position is held on the positioning element, and that the first shaft component and the second shaft component are in engagement with one another in the implantation position. This design makes it possible, in particular, to configure the tibial component modularly. In particular, the first shaft component may hereby always be of identical configuration. In a modular knee joint endoprosthesis system, the second shaft component may, for example, be provided in different lengths and/or having different diameters, in order to thus enable an optimal anchoring of the tibial component to the tibia of the patient.

The shaft can be easily configured in a modular manner if the first shaft component comprises a first connecting element, if the second shaft component comprises a second connecting element, and if the first and the second connecting element are in engagement in a force-locking and/or positive-locking and/or materially bonded manner in the implantation position. In particular, the two shaft components in engagement with one another in the implantation position are able to be transferred from a separation position into the implantation position by a force-locking and/or positive-locking and/or materially bonded connection with one another. In particular, this connection may be configured in such a way that it is no longer releasable. In particular, a stability of the tibial component can thus be improved.

The shaft can be formed in a simple manner if the one of the two connecting elements comprises an internal thread and if the other one of the two connecting elements comprises an external thread corresponding to the internal thread. The connecting elements configured as described may selectively be arranged or formed on the one or the other shaft component.

It is advantageous if the first connecting element is configured in the form of a nut and if the second connecting element is configured in the form of a threaded bolt portion projecting from the second shaft component. The two shaft components can then be coupled to one another by screwing the nut to the threaded bolt portion.

It is advantageous if the first connecting element is configured in the form of a screw having a screw head and a threaded bolt portion projecting from the screw head and if the second connecting element is configured in the form of a blind hole formed on the second shaft component. If, for example, the blind hole is provided with an internal thread, the screw can be screwed with the threaded bolt portion to the blind hole in order to connect the two shaft components to one another.

Advantageously, the screw head forms the joint projection. This makes it possible, in particular, to couple the screw, namely its screw head, to the positioning element in an articulated, in particular ball-jointed manner.

According to a further preferred embodiment, provision may be made that the positioning element comprises a connecting element receptacle for accommodating at least a portion of the first connecting element and comprises a connecting element perforation, and that the first or the second connecting element passes through the connecting element perforation in the implantation position. This configuration makes it possible, in particular, to couple the two shaft components to the positioning element in the implantation position, namely in particular in such a way that the positioning element is arranged between the second shaft component and one of the two connecting elements. In particular, a clamping connection between these can thus be established. In particular, the first connecting element in the implantation position can be completely accommodated in the connecting element receptacle. In particular, it can be prevented that the fixing element presses against the first connecting element in the implantation position.

In particular, to protect the first connecting element in the implantation position from any damage, it is advantageous if the fixing element closes the connecting element receptacle in the implantation position.

Further, it may be favorable if the connecting element receptacle comprises the joint projection seat and if the second positioning element part closes the connecting element receptacle. This enables, in particular, a compact structure of the tibial component, for example when an articulated connection between the shaft and the tibial plate is desired.

It is advantageous if a positioning device projection projects from the bottom side on the tibial plate and if the positioning element receptacle is formed at least partially in the positioning device projection. The positioning device projection makes it possible, in particular, to position the positioning element on the tibial plate in a defined manner and, in particular, also in a protected manner.

For a stable anchoring of the tibial component to the tibia that, in particular, is also secured against rotation, it is favorable if from the bottom side at least one stabilization projection is arranged or formed protruding laterally from the positioning device projection and pointing away from the bottom side of the tibial plate. In particular, two such stabilization projections may be provided. For example, they may be of mirror-symmetrical configuration relative to a symmetry plane containing the longitudinal axis. For example, the two stabilization projections may enclose between them an angle that is smaller than 180°.

It is advantageous if the at least one stabilization projection is of rectilinear or curved configuration. In particular, it may be of convexly curved configuration pointing in the anterior direction. Thus, an anchoring of the tibial plate on the tibia can be improved and, in particular, secured against a rotation relative to the tibia.

In order to provide optimal abutment faces, in particular for a meniscal component on the top side and for a planar prepared tibia, it is advantageous if the top side and/or the bottom side of the tibial plate is/are of planar or substantially planar configuration.

The top side is favorably configured in the form of a tibial joint face. For example, the tibial joint face can interact directly with a corresponding joint face of a femoral component. However, it is also possible to mount a meniscal component on the tibial joint face, said meniscal component being configured to cooperate with a femoral component of a knee joint endoprosthesis.

The object stated at the outset is further achieved, in accordance with the present disclosure, in a knee joint endoprosthesis system of the kind described at the outset in that the at least one tibial component is configured in the form of one of the tibial components described above.

Such a knee joint endoprosthesis system then has, in particular, the advantages described above in connection with preferred embodiments of tibial components. It is also possible, for example, to configure the knee joint endoprosthesis system modularly. For example, different tibial plates can be provided, which differ from one another in shape and/or size. This enables a surgeon to select the tibial component that is most suitable for a patient for implantation.

It is advantageous if the knee joint endoprosthesis comprises at least one meniscal component that is coupleable to the tibial component and if the meniscal component has a joint face that cooperates with the at least one femoral component. In particular, it is thus possible to arrange the meniscal component between the femoral component and the tibial component in order to thus form knee joint endoprostheses of different types. For example, the meniscal component may be immovably arranged or mounted on the tibia. However, it is also conceivable to movably couple the meniscal component to the tibial component. Here, all types of possible couplings and relative movements are conceivable, in particular pivoting movements and/or displacement movements of the meniscal component and the tibial component relative to one another. For example, the meniscal component may have a planar bottom side, which is configured to cooperate with a planar top side of the tibial component.

In order to be able to implant the knee joint endoprosthesis in a patient that is best suited for them, it is favorable if the knee joint endoprosthesis system comprises a plurality of shafts differing in their length and/or their cross section for selectively coupling to the positioning device of the tibial component. In particular, these shafts can be coupled in various ways with the positioning element of the positioning device. In particular, knee joint endoprostheses can thus be created, in which the shafts in the alignment position relative to the tibial plate are not only displaceable in parallel to the positioning plane, but also are pivotable on the tibial plate.

### BRIEF DESCRIPTION OF THE DRAWINGS

The subsequent description of preferred embodiments of the present disclosure serves in conjunction with the drawings for further explanation. In the drawings:

FIG. 1: shows a schematic perspective total view of an embodiment of a knee joint endoprosthesis of a knee joint endoprosthesis system, which replaces a degenerated knee joint of a patient;

FIG. 2: shows a schematic perspective partially broken exploded depiction of the embodiment depicted in FIG. 1 of a knee joint endoprosthesis;

FIG. 3: shows a schematic perspective exploded depiction of the embodiment depicted in FIG. 2 of a tibial component;

FIG. 4: shows a section view along line 4-4 in FIG. 3;

FIG. 5: shows a view analogous to FIG. 4, wherein the shaft is deflected out of a basic position relative to the tibial plate;

FIG. 6: shows a section view along line 6-6 in FIG. 4;

FIG. 7: shows a section view analogous to FIG. 6, wherein the shaft is deflected out of a basic position relative to the tibial plate;

FIG. 8: shows an enlarged view of section A in FIG. 6;

FIG. 9: shows a schematic section view analogous to FIG. 6 of a further embodiment of a tibial component;

FIG. 10: shows a schematic section view analogous to FIG. 9, wherein the shaft is deflected out of a basic position relative to the tibial plate;

FIG. 11: shows a schematic section view analogous to FIG. 9 of a further embodiment of a tibial component;

FIG. 12: shows a schematic section view analogous to FIG. 11, wherein the shaft is deflected out of a basic position relative to the tibial plate; and

FIG. 13: shows a schematic, partially broken plan view of a top side of a further embodiment of a tibial component.

### DETAILED DESCRIPTION

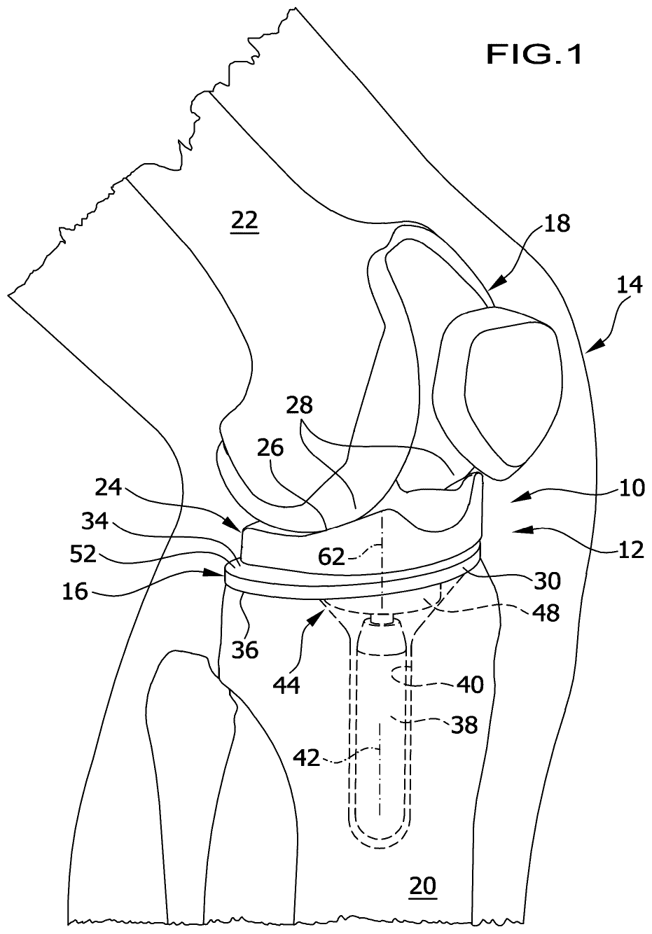

A first embodiment of a knee joint endoprosthesis 10 of a knee joint endoprosthesis system 12 is depicted schematically in FIG. 1. The knee joint endoprosthesis 10 is implanted as a replacement for a degenerated natural knee joint in the knee 14 of a patient.

The knee joint endoprosthesis 10 comprises a tibial component 16 and a femoral component 18 that cooperates therewith. The tibial component 16 is configured to anchor to a proximal, prepared end of a tibia 20. The femoral component 18 is configured to anchor to a distal, prepared end of a femur 22. The tibial component 16 and the femoral component 18 are configured corresponding to one another to form the knee joint endoprosthesis 10.

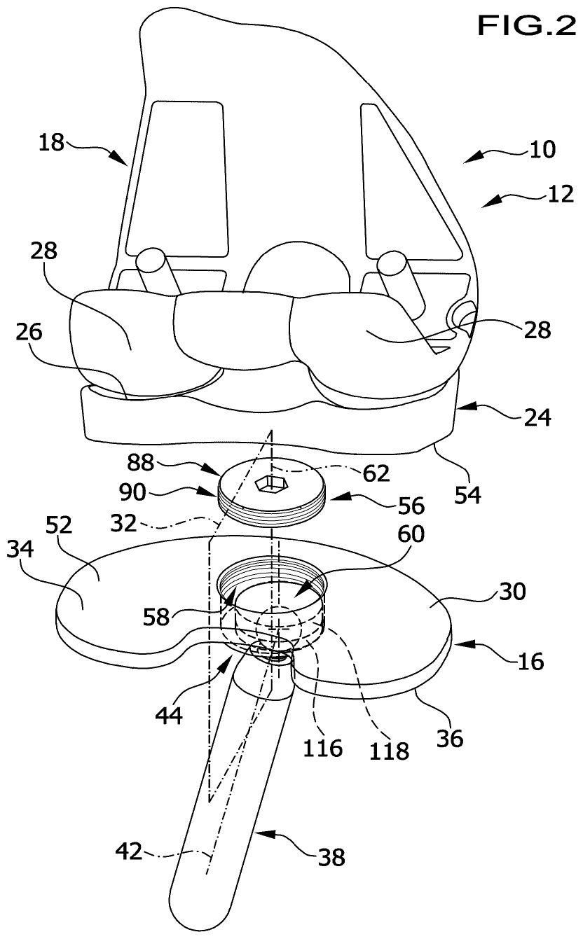

The embodiment of the knee joint endoprosthesis 10 depicted in FIGS. 1 and 2 comprises an optional meniscal component 24, which is arranged between the tibial component 16 and the femoral component 18. In the embodiment described in detail below, the meniscal component 24 is selectively able to be fixed to the tibial component 16 or is able to be moved relative thereto.

The meniscal component 24 has a joint face 26 that cooperates with the femoral component 18. This abuts against a condyle face 28 defined by the femoral component 18, wherein the condyle face 28 is able to roll on and/or slide along the joint face 26.

The tibial component 16 comprises a tibial plate 30, which in a plan view is of mirror symmetrical configuration relative to a symmetry plane 32 and is of substantially U-shaped or kidney-shaped configuration. The tibial plate 30 defines a top side 34 and a bottom side 36 pointing in the opposite direction.

A shaft 38 projects from the bottom side 36, said shaft being configured to anchor in the medullary space 40 of the tibia 20, which is drawn schematically in FIG. 1 with dashed lines, in order to fix the tibial component 16 to the tibia 20.

The shaft 38 is pivotable on the tibial plate 30 and/or about a longitudinal axis 42 defined by said shaft. This is described in more detail below.

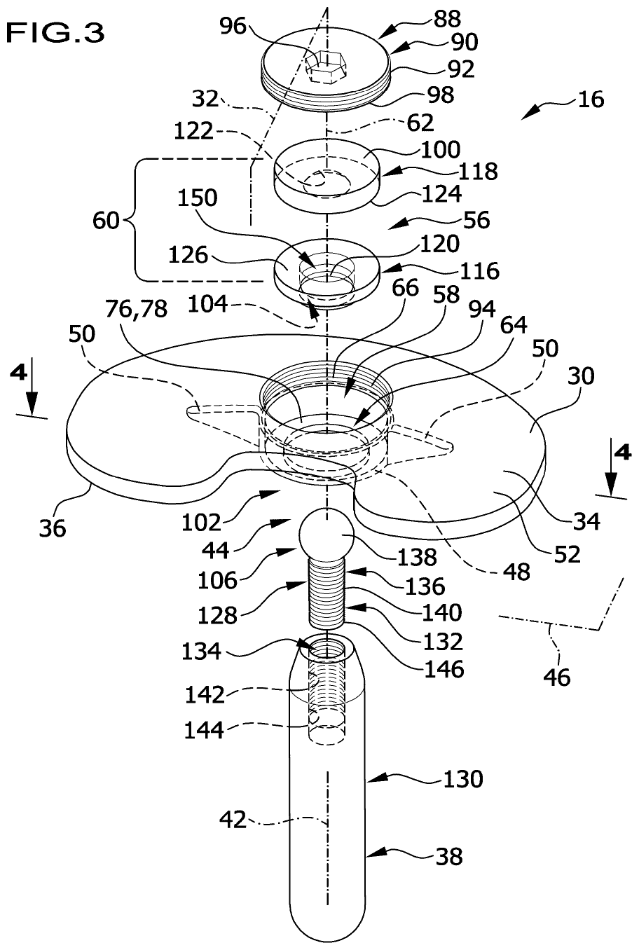

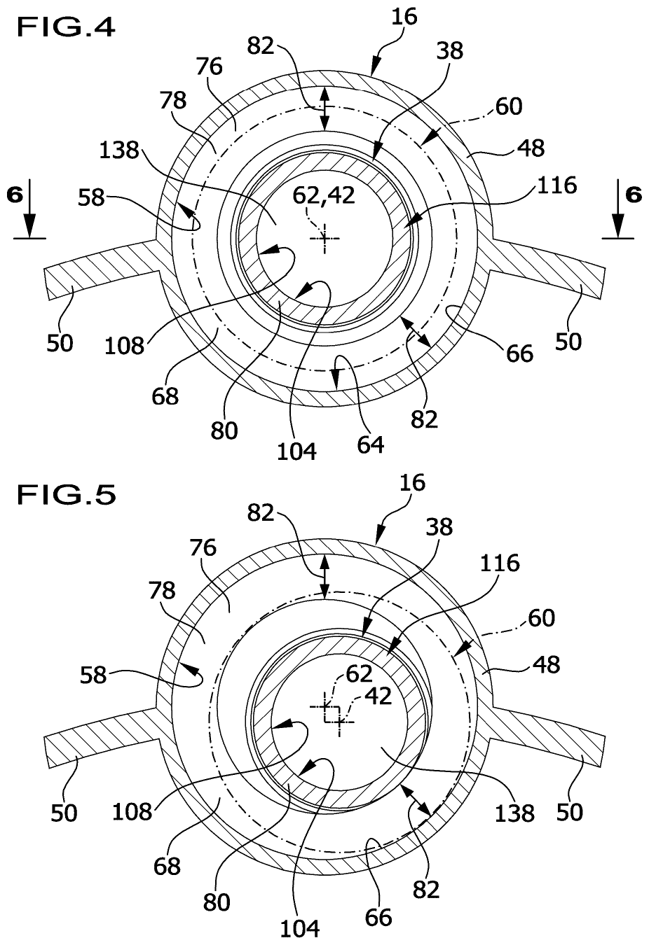

The tibial component 16 comprises a positioning device 44 for positioning the shaft 38 on the tibial plate 30 in a plurality of different positions. For this purpose, the positioning device 44 is configured in such a way that the shaft 38 is transferable from one of the different positions into another one of the different positions by a movement in parallel to a positioning plane 46 that extends in parallel or substantially in parallel to the top side 34. The embodiment of a tibial component 16 depicted schematically in FIGS. 1 to 8 comprises a positioning device projection 48 projecting from the bottom side 36 of the tibial plate 30. It forms part of the positioning device 44.

From the bottom side 36, two stabilization projections 50 protrude laterally from the positioning device projection 48 and away from the bottom side 36. In the embodiment depicted in the Figures, the stabilization projections 50 are of curved configuration, namely convexly curved pointing in the anterior direction.

In embodiments not depicted, the stabilization projections are of rectilinear configuration, in particular they may then extend in a plane perpendicular to the symmetry plane 32.

The top side 34 and the bottom side 36 of the tibial plate 30 are of planar or substantially planar configuration in the embodiment shown in FIGS. 1 to 8.

The top side is configured in the form of a tibial joint face 52. When the meniscal component 24 is movably mounted on the tibial component 16, said tibial joint face 52 forms a sliding pairing with a bottom side 54 of the meniscal component 24.

The tibial component 16 further comprises a fixing device 56 for fixing the shaft 38 in an implantation position, as shown schematically in FIG. 1. The fixing device 56 is transferable from an alignment position, as is schematically depicted in FIG. 2, into the implantation position.

In the alignment position, the shaft 38 is movable relative to the tibial plate 30 in parallel to the positioning plane 46. In the embodiment shown in FIGS. 1 to 8, the shaft 38 is displaceable relative to the tibial plate 30 in parallel to the positioning plane 46 and is also pivotable relative thereto, as is explained in the following.

The positioning device 44 comprises a positioning element receptacle 58 formed on the tibial plate 30. This is partially formed in the positioning device projection 48.

A positioning element 60 of the positioning device 44 is accommodated in the positioning element receptacle 58. The positioning element 60 and the shaft 38 are configured to be coupleable to one another. Furthermore, the positioning element receptacle 58 defines a longitudinal axis 62 extending perpendicularly to the positioning plane 46, which longitudinal axis lies in the symmetry plane 32.

The positioning element receptacle 58 is also configured and dimensioned in such a way that the positioning element 60 in the alignment position is displaceable relative to the positioning element receptacle 58 in parallel to the positioning plane 46.

In that embodiment, the positioning element receptacle 58 is configured and dimensioned in such a way that in each of the different positions that the positioning element 60 can adopt relative to the tibial plate 30 in the positioning element receptacle 58, said positioning element 60 is secured against a movement away from the tibial plate 30, namely in a direction pointing away from the bottom side 36. This is achieved, in particular, by a movement delimiting device 64. This is configured to delimit a movement of the positioning element 60 relative to the positioning element receptacle 58 in a direction transverse to top side 34, namely perpendicular to the top side in the embodiment depicted in FIGS. 1 to 8, away from the bottom side 36.

On the tibial plate 30, a perforation 66 is formed, which comprises the positioning element receptacle 58. The perforation 66 comprises a stop 68 that cooperates with the positioning element 60 and acts in the direction toward the top side 34.

The stop 68 is formed by a wall 70, which delimits the positioning element receptacle at the bottom side 36.

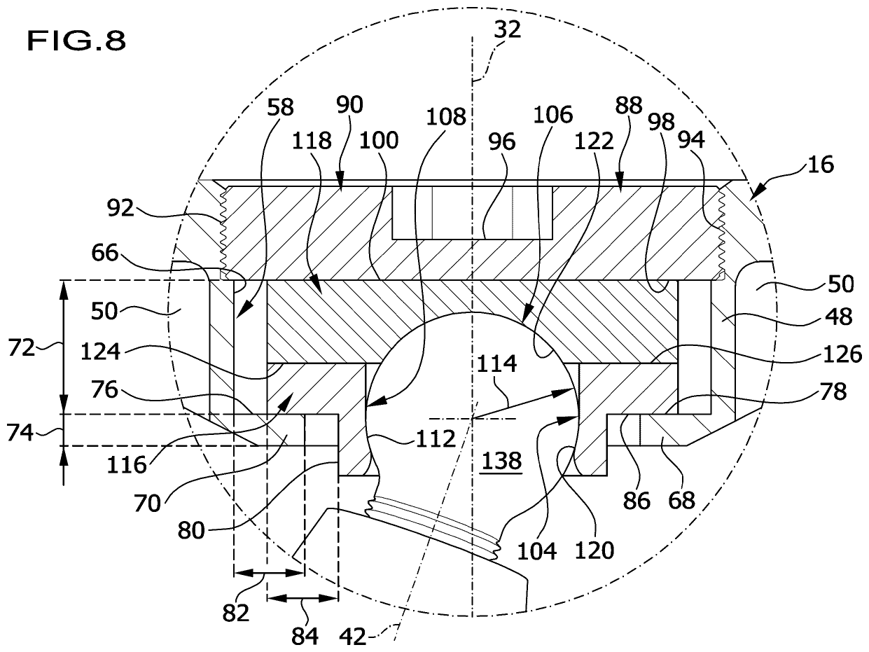

The perforation 66 comprises a receiving portion 72 defining the positioning element receptacle 58 and a perforation portion 74 passing through the wall 70. A cross sectional area of the receiving portion 72 relative to the longitudinal axis 62 is greater than a cross sectional area of the perforation portion 74. In this way, a stop face 76, which, as can be seen well particularly in FIG. 8, is planar, is defined by a wall face 78 of the wall 70 pointing in the direction toward the top side 34. In the embodiment depicted in FIGS. 1 to 8, the stop face 76 is of annular configuration.

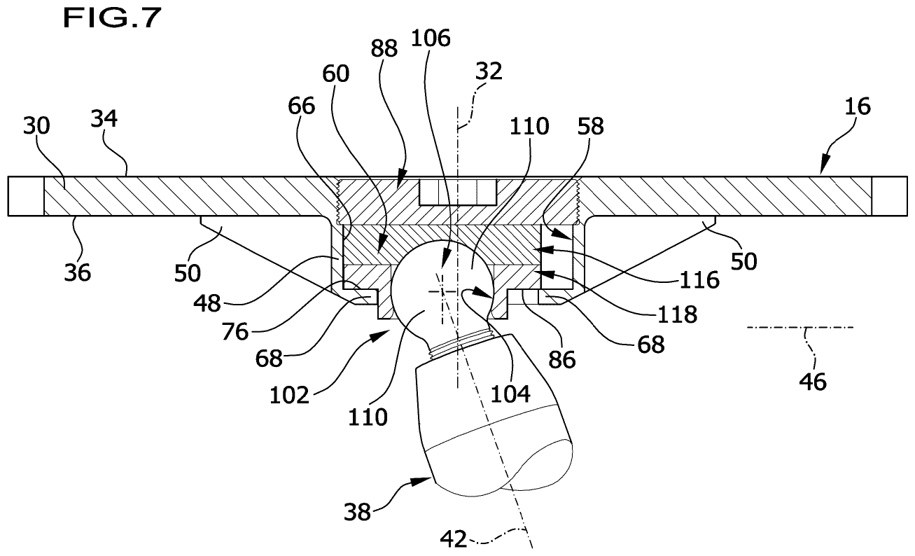

The positioning element 60 comprises a positioning element projection 80 engaging into or passing through the perforation portion 74. A cross sectional area of the positioning element projection 80 relative to the longitudinal axis 62 is smaller than a cross sectional area of the perforation portion 74. The dimensioning described makes it possible to displace the positioning element 60 in the positioning element receptacle 58 in parallel to the positioning plane 46, namely until the positioning element projection 80 strikes against the wall 70, as is schematically depicted in FIG. 7. The positioning element projection 80 in cooperation with the perforation portion 74 thus delimits a movement of the positioning element 60 perpendicular to the longitudinal axis 62.

In order to prevent the positioning element 60 from being able to pass through the perforation 66 in the region of the perforation portion 74, a width 82 of the stop face 76 is smaller than a width 84 of an annular face 86 of the positioning element 60 abutting against the abutment face 76.

In the embodiment depicted in FIGS. 1 to 8, a first cross sectional shape of the receiving portion 72 and a second cross sectional shape of the perforation portion 74 are geometrically similar. In this embodiment, both cross sectional shapes are circular.

In alternative embodiments, the first and second cross sectional shapes differ from one another. In particular, the first and/or the second cross sectional shape may be oval or polygonal. In particular, a polygonal cross sectional shape may be rectangular or square.

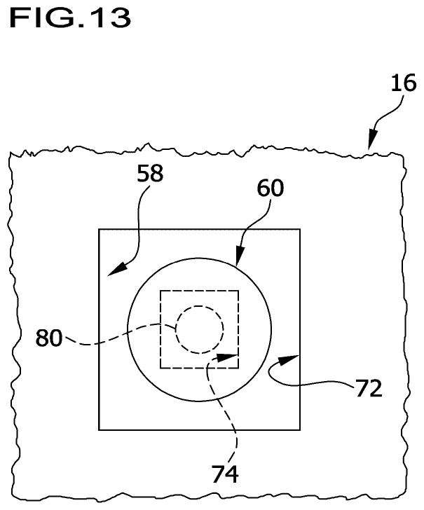

Schematically depicted in FIG. 13 is a plan view of an embodiment of a tibial component 16 in which the receiving portion 72 and the perforation portion 74 are geometrically similar and each have a square cross sectional shape. The positioning element 60, on the other hand, has a circular cross sectional shape, as well as the positioning element projection 80.

In an analogous manner, as illustrated, e.g., in FIG. 13, cross sectional shapes of the positioning element 60, the positioning element projection 80, the receiving portion 72, and the perforation portion 74 can be combined with one another in almost any manner. Ultimately, it must only be ensured that the positioning element 60 is dimensioned in cross section such that it is movable in the positioning element receptacle 58 in parallel to the positioning plane 46 in the alignment position and is not able to pass through the perforation portion 74 in any position. Accordingly, the outer dimensions of the positioning element projection 80 and of a free cross sectional area of the perforation portion 74 must also be dimensioned to make this possible.

In the embodiment depicted in FIGS. 1 to 8, the fixing device 56 is configured to immovably fix the positioning element 60 in the positioning element receptacle 58 in the implantation position. In order to achieve this, the fixing device 56 comprises a fixing element 88, which in the implantation position directly abuts against the positioning element 60 and in the implantation position holds the positioning element 60 in the positioning element receptacle 58 in a clamping manner, namely clamped between the fixing element 88 and the stop 68.

The fixing element 88 comprises a screw element 90 with an external thread 92. Commencing from the top side 34 of the tibial plate 30, the perforation 66 is provided with an internal thread 94 corresponding to the external thread 92. As a result of this configuration, the disc-shaped fixing element 88 can be screwed into the perforation 66 from above. In order to facilitate this, a tool element receptacle 96 in the form of a polygonal socket is formed on the fixing element 88, said polygonal socket being open pointing away from the top side 34. A screw-in tool with a tool end in the form of an external polygon corresponding to the tool element receptacle 96 can thus be used to screw the fixing element 88 into the perforation 66.

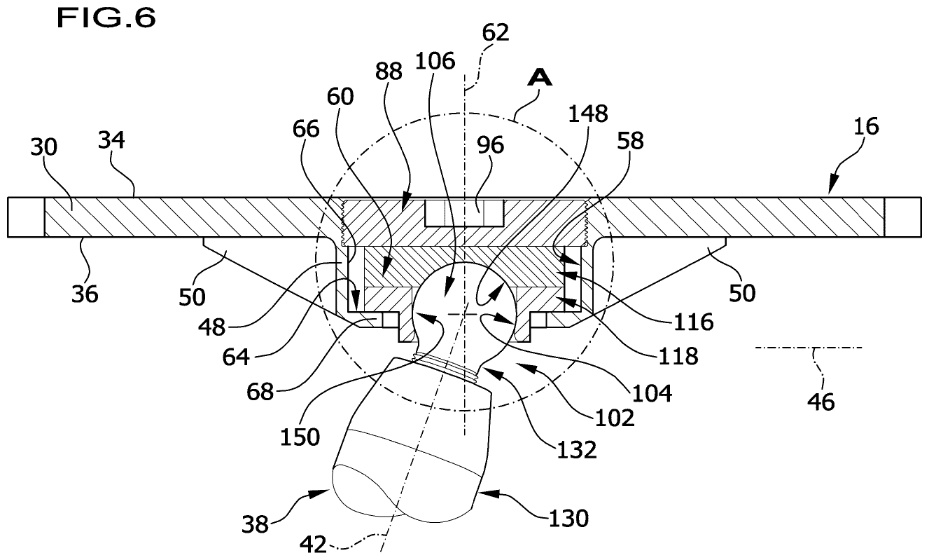

As schematically depicted in FIGS. 6 to 8, the fixing element 88 closes the perforation 66 in the implantation position.

The fixing element 88 has a fixing element clamping face 98, which in the implantation position is held against the positioning element 60 in a clamping manner. Here, it abuts against a fixing element abutment face 100 of the positioning element 60.

In the embodiment of FIGS. 1 to 8, the shaft 38 is mounted on the tibial plate 30 so as to be pivotable and so as to be rotatable about the longitudinal axis 42 of the shaft 38. However, the shaft 38 is pivotable relative to the tibial plate 30 only in the alignment position.

The shaft 38 is arranged on the tibial plate 30 in the region of the symmetry plane 32. It can be displaced in the alignment position in parallel to the positioning plane 46 such that the longitudinal axis 42 of the shaft 38 no longer coincides with the longitudinal axis 62 of the positioning device 44 or the longitudinal axis 42 is deflected out of the symmetry plane 32 and is inclined relative thereto.

In a basic position of the tibial component 16, the longitudinal axis 42 and the longitudinal axis 62 coincide.

In order to be able to anchor the tibial component 16 in the medullary space 40 of the tibia 20 in a simple manner, the shaft is arranged in a middle or central region of the bottom side 36 of the tibial plate 30.

The shaft 38 is of rotationally symmetrical or substantially rotationally symmetrical configuration relative to its longitudinal axis 42.

For pivoting the shaft 38 relative to the tibial plate 30, said shaft is mounted on the tibial plate 30 in an articulated manner. In the embodiment depicted in FIGS. 1 to 8, a ball-jointed mounting is provided. Alternatively, in an embodiment not shown, the shaft 38 is mounted on the tibial plate 30 in a hinge-jointed manner.

For mounting the shaft 38 on the tibial plate 30 in an articulated manner, the tibial component 16 comprises a joint device 102. The joint device 102 comprises a first joint element 104 and a second joint element 106. The first joint element 104 is arranged or formed on the tibial plate 30. The second joint element 106 is arranged or formed on the shaft 38.

The first joint element 104 and the second joint element 106 are formed corresponding to one another and are in engagement with one another in an articulated manner. In the embodiment depicted schematically in FIGS. 1 to 8, the first joint element 104 is configured in the form of a joint receptacle 108. The second joint element 106 is configured in the form of a joint projection 110 engaging into the joint receptacle 108.

In the embodiment schematically depicted in FIGS. 1 to 8, the joint projection 110 is of spherical configuration. The joint receptacle 108 comprises a hollow-spherical abutment face region 112 for the joint projection 110.

A radius 114 defined by the abutment face region 112 corresponds to a radius of the spherical joint projection 110.

As can be seen, in particular, in FIGS. 6 and 8, the positioning element 60 comprises the joint receptacle 108. This configuration enables a ball-jointed connection of the shaft 38 to the positioning element 60 and thus also to the tibial plate 30.

In the embodiment of FIGS. 1 to 8, the positioning element 60 is of two-part configuration and comprises a first positioning element part 116 and a second positioning element part 118.

The first positioning element part 116 comprises a joint projection seat 120 for the joint projection 110. The second positioning element part 118 is arranged between the first positioning element part 116 and the fixing element 88. Further, the second positioning element 118 abuts against the joint projection 110, namely with a hollow-spherical depression 122, the radius of which corresponds to the radius 114 of the joint projection 110. The depression 122 is surrounded by an annular abutment face 124, which abuts against an annular abutment face 126 of the first positioning element part 116. The abutment faces 124 and 126 extend in parallel to the positioning plane 46.

In the described embodiment, the second positioning element part 118 comprises the fixing element abutment face 100.

In the embodiment of FIGS. 1 to 8, the joint receptacle 108 is of rotationally symmetrical configuration, namely relative to the longitudinal axis 62, which defines a surface normal of the bottom side 36 of the tibial plate 30.

In the embodiment of FIGS. 1 to 8, the shaft 38 is of multi-part configuration. It comprises a first shaft component 128 and a second shaft component 130. The first shaft component 128 in the implantation position is held on the positioning element 60. In the implantation position, the two shaft components 128 and 130 are in engagement with one another, namely in a force-locking and/or positive-locking and/or materially bonded manner.

For establishing a connection between the shaft components 128 and 130, a first connecting element 132 is arranged or formed on the first shaft component 128. The second shaft component 130 comprises a second connecting element 134. In the connecting position, the two connecting elements 132 and 134 are in engagement in a force-locking and/or positive-locking and/or materially bonded manner.

In the embodiment of FIGS. 1 to 8, the first connecting element 132 is configured in the form of a screw 136 having a screw head 138 and a threaded bolt portion 140 projecting from said screw head. The second connecting element 134 is configured in the form of a blind hole 142 formed on the second shaft component 130. The blind hole 142 has an internal thread 144, which corresponds to an external thread 146 of the threaded bolt portion 140. The blind hole 142 is formed coaxially with the longitudinal axis 42. In the embodiment of FIGS. 1 to 8, the screw head 138 forms the joint projection 110.

The positioning element 60 comprises a connecting element receptacle 148. It is configured to accommodate a portion of the first connecting element 132, namely the joint projection 110.

The positioning element 60 further comprises a connecting element perforation 150. The first connecting element 132 passes through this connecting element perforation 150 in the implantation position, namely with a portion of the joint projection 110 or the threaded bolt portion 140.

In the embodiment of FIGS. 1 to 8, the connecting element receptacle 148 comprises the joint projection seat 120. The second positioning element part 118 closes the connecting element receptacle 148 in the implantation position.

The functioning of the knee joint endoprosthesis system 12 is explained in the following in more detail in connection with FIGS. 1 to 8.

The tibia 20 and the femur 22 are prepared to replace the damaged knee joint of a patient. Tibial and femoral components 16, 18 adapted to the physiological situation of the patient are selected and anchored to the bones.

To optimally adapt the selected tibial component 16, the tibial plate 30 is first placed against the prepared bone face of the tibia in order to determine the optimal size. If necessary, a tibial plate 30 that is better suited to the shape and size of the tibia 20 is selected.

Now, the first positioning element 116 is inserted into the positioning element receptacle 58, namely in such a way that the positioning element projection 80 formed on the first positioning element part 116 engages into the perforation portion 74. Now, the first connecting element 132 can be inserted with the threaded bolt portion 140 into the positioning element receptacle 58 and passed through the connecting element perforation 150 until the spherical joint projection 110 abuts against the joint projection seat 120.

The second positioning element 118 can now be placed with the depression 122 against the joint projection 110. The fixing element 88 can then be screwed into the perforation 66. However, the fixing element 88 is not screwed in so far that no movement between the positioning element 60 is possible. In this alignment position, in which the positioning element 60 is displaceable in the positioning element receptacle 58 in parallel to the positioning plane 46, the first connecting element 132 can now be positioned and aligned in the desired manner on the tibial plate 30.

A second shaft component 130 suited to the medullary space 40 in length and diameter is selected from the modular knee joint endoprosthesis system 12 and connected to the first shaft component 128.

When the shaft 38 coupled to the tibial plate 30 in the alignment position in that way is aligned in the desired manner, i.e. in particular pivoted out of the symmetry plane 32 and optionally displaced relative thereto, the fixing element 88 is further screwed in until it presses the second positioning element part 118 against the joint projection 110 and presses the latter against the joint projection seat 120 in a clamping manner. The tibial component now adopts the implantation position. The shaft 38 is immovably held on the tibial plate 30.

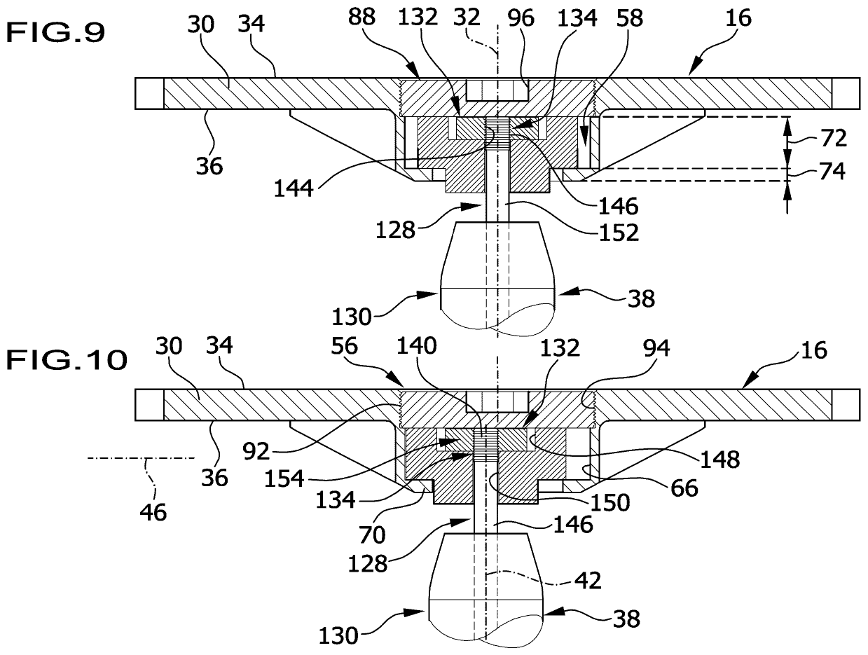

Schematically depicted in FIGS. 9 to 12 are two further embodiments of tibial components 16 of a knee joint endoprosthesis system 12. Due to the great similarities in the structural design of the embodiments with the embodiment of a tibial component 16 shown schematically in FIGS. 1 to 8, identical or functionally comparable elements are provided with the same reference numerals as in the embodiment of FIGS. 1 to 8.

The embodiments of FIGS. 9 to 12 differ from the embodiment of the tibial component of FIGS. 1 to 8 in the design of the positioning element 60, the shaft 38 with the shaft components 128 and 130, and the connecting elements 132 and 134.

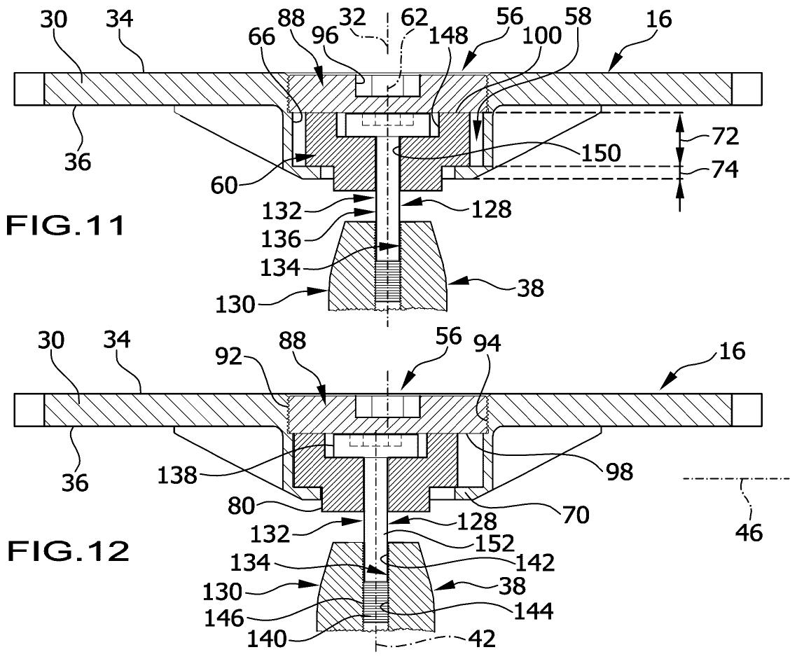

It should also be noted that the shaft 38 in the embodiments of FIGS. 9 to 12 is not pivotable on the tibial plate 30 in the alignment position. It is only displaceable in parallel to the positioning plane 46, in particular starting from a basic position, in which the longitudinal axis 42 lies in the symmetry plane 32 of the tibial plate 30, into a deflected position, in which the longitudinal axis 42 is displaced in parallel to the symmetry plane 32. FIGS. 9 and 11 each show the basic positions schematically. Deflected positioned are depicted in FIGS. 10 and 12.

The embodiment of the tibial component 16 according to FIGS. 1 to 8 has the greatest similarity with the embodiment of FIGS. 11 and 12. Here, too, the first connecting element 132 is configured in the form of a screw 136. However, the positioning element 60 is of one-piece configuration and has a connecting element receptacle 148, which forms a recess in the positioning element 60 starting from the fixing element abutment face 100. The connecting element receptacle 148 is dimensioned such that it is able to accommodate the screw head 138 of the screw 136.

A connecting element perforation 150 in the form of a bore is formed on the positioning element 60 coaxially with the longitudinal axis 42. It is passed through by a shank portion 152 of the screw 136. The shank portion 152 has no external thread. In the region of a distal end of the shank portion 152, said shank portion is configured in the form of a threaded bolt portion 140, which has an external thread that corresponds to an internal thread 144 of a blind hole 142 in the second shaft component 130. Thus, the first connecting element 132 in the form of the screw 136 forms the first shaft component 128.

For mounting the tibial component 16, the positioning element 60 is first introduced from the top side 34 of the tibial plate into the positioning element receptacle 58, namely in such a way that the positioning element projection 80 engages into the perforation portion 74. Now the screw 136 can be pushed with the threaded bolt portion 140 in front through the connecting element perforation 150 until the screw head 138 is received in the connecting element receptacle 148.

The second shaft component 130 can now be screwed to the screw 136. Finally, the fixing element 88 is screwed into the internal thread 94, which is formed at the perforation 66, until the fixing element clamping face 98 abuts against the fixing element abutment face 100. In this way, as in the embodiment of FIGS. 1 to 8, the positioning element 60 can then be held between the fixing element 88 and the wall 70 in a clamping manner.

In order to align the shaft 38 on the tibial plate 30, only the fixing element 88 is loosened somewhat so that the positioning element 60 in the positioning element receptacle 58 can be displaced in parallel to the positioning plane 46, for example from the basic position, which is shown schematically in FIG. 11, into a deflected position, as it is shown schematically in FIG. 12.

The embodiment of the tibial component 16 as shown schematically in FIGS. 9 and 10 differs from the embodiment of FIGS. 11 and 12 only in the design of the shaft components 128 and 130 and the connecting elements 132 and 134. Therefore, only these differences are explained in the following.

The second shaft component 130 does not have a blind hole, but instead a shank portion 152 projects therefrom pointing in the direction toward the tibial plate 30, a threaded bolt portion 140 being formed at the distal end of said shank portion 152. The first connecting element 132 is configured in the form of a nut 154, which has an internal thread 144 that corresponds to the external thread 146 of the threaded bolt portion 140. Thus, the nut 154 also forms the first shaft component 128.

For implanting the tibial component 16 according to the embodiment of FIGS. 9 and 10, in a first step, the one-piece, monolithically formed positioning element 60, which is of identical configuration to the positioning element 60 according to the embodiment of FIGS. 11 and 12, is inserted with the positioning element projection 80 into the positioning element receptacle 58 engaging into the perforation portion 74.

Now, from below, i.e. in the direction toward the bottom side 36, the threaded bolt portion 140 can be pushed through the connecting element perforation 150 until the threaded bolt portion 140 is positioned in the region of the connecting element receptacle 148. To connect the second shaft component 130 to the positioning element 60, the nut 154 is now screwed to the threaded bolt portion 140.

The shaft 38 coupled to the positioning element 60 can now be displaced in parallel to the positioning plane 46. In order to fix a desired position of the shaft 38 relative to the tibial plate 30, as in the other embodiments, the fixing element 88 is screwed into the perforation 66 in order to hold the positioning element 60 between said fixing element and the wall 70 in a clamping manner.

As mentioned, the described embodiments of knee joint endoprostheses 10 also enable, in particular, a modular configuration. Here, in particular, different second shaft components 130 may be provided, which differ in length and/or diameter and/or shape from one another, so that a surgeon can select the shaft component that is most appropriate for a patient in order to be able to ensure an optimal fit of the same in the medullary space 40.

All described tibial components 16 allow the shaft 38 to be aligned and fixed in different positions relative to the tibial plate 30. In the embodiment of FIGS. 1 to 8, in particular, a pivoting of the shaft 38 about a center point of the joint projection 110 is also possible.

### REFERENCE NUMERAL LIST

10 knee joint endoprosthesis
12 knee joint endoprosthesis system
14 knee
16 tibial component
18 femoral component
20 tibia
22 femur
24 meniscal component
26 joint face
28 condyle face
30 tibial plate
32 symmetry plane
34 top side
36 bottom side
38 shaft
40 medullary space
42 longitudinal axis
44 positioning device
46 positioning plane
48 positioning device projection
50 stabilization projection
52 tibial joint face
54 bottom side
56 fixing device
58 positioning element receptacle
60 positioning element
62 longitudinal axis
64 movement delimiting device
66 perforation
68 stop
70 wall
72 receiving portion
74 perforation portion
76 stop face
78 wall face
80 positioning element projection
82 width
84 width
86 annular face
88 fixing element
90 screw element
92 external thread
94 internal thread
96 tool element receptacle
98 fixing element clamping face
100 fixing element abutment face
102 joint device
104 first joint element
106 second joint element
108 joint receptacle
110 joint projection
112 abutment face region
114 radius
116 first positioning element part
118 second positioning element part
120 joint projection seat
122 depression
124 abutment face
126 abutment face
128 first shaft component
130 second shaft component
132 first connecting element
134 second connecting element
136 screw
138 screw head
140 threaded bolt portion
142 blind hole
144 internal thread
146 external thread
148 connecting element receptacle
150 connecting element perforation
152 shank portion
154 nut

1. A tibial component for a knee joint endoprosthesis, the tibial component comprising:
a) a tibial plate with a top side and a bottom side;
b) a shaft projecting from the bottom side for insertion into a medullary space of a tibia for anchoring the tibial component to the tibia; and
c) a positioning device for positioning the shaft on the tibial plate in different positions,
the positioning device configured such that the shaft is transferable from a first position of the different positions into a second position of the different positions by a movement in parallel to a positioning plane that extends parallel to the top side.

2. The tibial component according to claim 1, further comprising a fixing device for fixing the shaft in an implantation position, the fixing device being transferable from an alignment position, in which the shaft is movable relative to the tibial plate in parallel to the positioning plane, into the implantation position, in which the shaft is immovably held on the tibial plate.

3. The tibial component according to claim 2, wherein the positioning device comprises a positioning element receptacle formed on the tibial plate and a positioning element arranged in the positioning element receptacle, wherein the positioning element and the shaft are configured to be coupleable to one another and wherein the positioning element receptacle defines a longitudinal axis extending perpendicularly to the positioning plane and is configured and dimensioned such that the positioning element in the alignment position is displaceable relative to the positioning element receptacle in parallel to the positioning plane.

4. The tibial component according to claim 3, wherein:
a) the positioning element receptacle is configured and dimensioned such that the positioning element is secured in each of the different positions against a movement away from the tibial plate in a direction pointing away from the bottom side,
and/or
b) the positioning device comprises a movement delimiting device for delimiting a movement of the positioning element relative to the positioning element receptacle in a direction transverse to the top side away from the bottom side,
and/or
c) a perforation is formed on the tibial plate, wherein the perforation comprises the positioning element receptacle, and wherein the perforation comprises a stop that cooperates with the positioning element and acts in the direction toward the top side.

5. The tibial component according to claim 4, wherein the positioning element receptacle is delimited toward the bottom side by a wall, and wherein the perforation comprises a receiving portion defining the positioning element receptacle and a perforation portion passing through the wall.

6. The tibial component according to claim 5, wherein:
a) a cross sectional area of the receiving portion relative to the longitudinal axis is greater than a cross sectional area of the perforation portion,
and/or
b) the wall forms the stop,
and/or
c) the wall defines an annular stop face pointing in the direction toward the top side,
and/or
d) the positioning element comprises a positioning element projection engaging into or passing through the perforation portion,
and/or
e) a first cross sectional shape of the receiving portion and a second cross sectional shape of the perforation portion are geometrically similar or differ from one another.

7. The tibial component according to claim 3, wherein the fixing device is configured to immovably fix the positioning element in the positioning element receptacle in the implantation position.

8. The tibial component according to claim 4, wherein the fixing device comprises a fixing element and wherein the fixing element abuts directly or indirectly against the positioning element and in the implantation position holds the positioning element in the positioning element receptacle in a clamping manner.

9. The tibial component according to claim 8, wherein:
a) the positioning element in the implantation position is held between the fixing element and the stop in a clamping manner,
and/or
b) the fixing element comprises a screw element with an external thread and wherein the perforation comprises an internal thread corresponding to the external thread commencing from the top side of the tibial plate,
and/or
c) the fixing element closes the perforation in the implantation position,
and/or
d) the fixing element has a fixing element clamping face, which in the implantation position is held against the positioning element in a clamping manner,
and/or
e) the positioning element has a fixing element abutment face, which in the implantation position abuts against the fixing element.

10. The tibial component according to claim 1, wherein the shaft:
a) is mounted on the tibial plate so as to be pivotable and/or rotatable about a longitudinal axis of the shaft,
and/or
b) is arranged or formed in a region of a symmetry plane of the tibial plate,
and/or
c) is arranged or formed in a middle or central region of the bottom side of the tibial plate,
and/or
d) is of rotationally symmetrical or substantially rotationally symmetrical configuration relative to its longitudinal axis,
and/or
e) is mounted on the tibial plate in an articulated manner.

11. The tibial component according to claim 1, further comprising a joint device with a first joint element and a second joint element, wherein the first joint element is arranged or formed on the tibial plate, and wherein the second joint element is arranged or formed on the shaft, and wherein the first joint element and the second joint element are in engagement with one another in an articulated manner.

12. The tibial component according to claim 11, wherein the first joint element is configured as a joint receptacle and wherein the second joint element is configured as a joint projection engaging into the joint receptacle.

13. The tibial component according to claim 3, wherein the shaft is of multi-part configuration and comprises a first shaft component and a second shaft component, wherein the first shaft component in the implantation position is held on the positioning element, and wherein the first shaft component and the second shaft component are in engagement with one another in the implantation position.

14. The tibial component according to claim 13, wherein the first shaft component comprises a first connecting element, wherein the second shaft component comprises a second connecting element, and wherein the first and the second connecting element are in engagement in a force-locking and/or positive-locking and/or materially bonded manner in the implantation position.

15. The tibial component according to claim 14, wherein the positioning element comprises a connecting element receptacle for accommodating at least a portion of the first connecting element and comprises a connecting element perforation, and wherein the first or the second connecting element passes through the connecting element perforation in the implantation position.

16. The tibial component according to claim 3, wherein a positioning device projection projects from the bottom side on the tibial plate, and wherein the positioning element receptacle is formed at least partially in the positioning device projection.

17. The tibial component according to claim 1, wherein:
a) the top side and/or the bottom side of the tibial plate are of planar configuration,
and/or
b) the top side is configured as a tibial joint face.

18. A knee joint endoprosthesis system comprising:
at least one tibial component according to claim 1, for anchoring to a proximal end of a tibia; and
at least one femoral component for anchoring to a distal end of a femur and,
the at least one tibial component and the at least one femoral component corresponding to one another to form a knee joint endoprosthesis.

19. A tibial component for a knee joint endoprosthesis, the tibial component comprising:
a) a tibial plate with a top side and a bottom side;
b) a shaft projecting from the bottom side for insertion into a medullary space of a tibia for anchoring the tibial component to the tibia;
c) a positioning device for positioning the shaft on the tibial plate in different positions, wherein the positioning device comprises:
a positioning element receptacle formed on the tibial plate, the positioning element receptacle having a wall with a first opening through which the shaft extends and a second opening opposite the first opening, and
a positioning element arranged in the positioning element receptacle and operatively connected to the shaft,
wherein the positioning element receptacle and the first opening are dimensioned such that the positioning element and the shaft are movable relative to the tibial plate in any direction parallel to a positioning plane that extends parallel to the top side; and
d) a fixing device configured to be received in the second opening of the positioning element receptacle with the positioning element between the fixing device and the wall, the fixing device being movable from an alignment position, in which the positioning element and the shaft are movable relative to the tibial plate in parallel to the positioning plane, and an implantation position, in which the fixing device secures the positioning element and the shaft against movement relative to the tibial plate.

20. The tibial component according to claim 19, wherein:
the positioning element comprises a hollow-spherical abutment face;
the shaft comprises a spherical projection configured to fit into the hollow-spherical abutment face to provide relative pivoting movement between the shaft and the positioning element when the fixing device is in the alignment position; and
the fixing device is configured, when in the implantation position, to press the spherical projection against the hollow-spherical abutment face to prevent relative pivoting movement between the shaft and the position element.
